(19) United States
(12) Patent Application Publication
(10) Pub. No.: US 2019/0083384 A1
(43) Pub. Date: Mar. 21, 2019

(54) METHOD FOR CONDITIONING SKIN BY USING EARTHWORM PROTEIN

(71) Applicant: TCI CO., LTD., Taipei (TW)

(72) Inventors: Yung-Hsiang LIN, Taipei (TW); Yu-Ting LIN, Taipei (TW)

(21) Appl. No.: 15/853,070

(22) Filed: Dec. 22, 2017

(30) Foreign Application Priority Data
Sep. 19, 2017 (TW) 106132142

(51) Int. Cl. A61K 8/98; A61Q 19/00; A61K 8/64; A61K 8/66

(52) U.S. Cl. CPC A61K 8/987; A61K 8/66; A61K 8/64; A61Q 19/00

(57) ABSTRACT

A method for conditioning skin is provided, which comprises applying to a subject in need an effective amount of a composition, wherein the composition comprises earthworm protein. The method is especially for reducing the concentration of erythema on skin and/or decreasing the size of pores on skin.

### PRIORITY CLAIM

This application claims priority to Taiwan Patent Application No. 106132142, filed on Sep. 19, 2017, which is hereby incorporated by reference in its entirety.

### FIELD OF THE INVENTION

The present invention relates to the uses of earthworm protein, especially the uses of earthworm protein in conditioning skin.

### BACKGROUND OF THE INVENTION

Due to the intensive and high development in industries, the preparation processes of various traditional or high-tech manufacture industries would inevitably bring considerable pollutions to the environment. In the respect, the air pollution has been known as one of the primary reasons that causes cancers. In addition to forming a threat to human health, the air pollution causes non-ignorable damage to human skin.

The air pollution refers to not only the toxic gases, but also the particulate molecules having various sizes in the air. The toxic gases or particulate molecules are highly irritative to the skin and easily cause inflammation and erythredema on the skin. Furthermore, the toxic gases and particulate molecules, due to their toxicity or small size, can deeply penetrate into the bottom layer of skin and seriously damage the original skin barrier, and thus, may cause skin sensitivity and many uncomfortable symptoms such as dry, pruritus, and fever.

Earthworm (Pheretima), a traditional medicinal herbal, is generally considered to be effective in promoting blood circulation, dispersing blood stasis and preventing cardiovascular diseases. Earthworm protein, an extract of earthworms, contains trace elements such as iron, manganese, copper, zinc, and selenium and is rich in various peptides and enzymes. It is known that collagenase, plasmin, and lumbrokinase contained in the earthworm protein are effective in dissolving thrombus to a certain extent and thus can be used for preventing cardiovascular diseases or preventing/alleviating stroke sequelae. However, the specific effect of other ingredients in earthworm protein is still under investigation. Also, the effects of earthworm protein in conditioning skin have never been studied.

### SUMMARY OF THE INVENTION

An objective of the present invention is to provide a use of earthworm protein in conditioning skin. As illustrated by the examples provided hereinafter, earthworm protein in accordance with the present invention can reduce the concentration of erythema in skin.

Another objective of the present invention is to provide a use of earthworm protein in decreasing the size of pores on skin. As illustrated by the examples provided hereinafter, earthworm protein in accordance with the present invention can decrease the size of pores on skin.

To achieve the aforementioned objectives, the present invention provides a composition comprising earthworm proteins, and a method comprising applying to a subject in need an effective amount of the aforementioned composition to reduce the concentration of erythema in skin and/or decrease the size of pores on skin.

In the use in accordance with the present invention, the composition could be applied to the subject in need as a solution, an emulsion, or a paste, but is not limited thereby.

In one embodiment of the present invention, the composition is adsorbed on a mask and is applied via the mask.

In one embodiment of the present invention, the concentration of the earthworm protein (as dry powders) in the composition is about 0.005 to 0.05 wt %.

In the use in accordance with the present invention, the composition is placed in a high-pressure jet bottle and is applied as a spray.

In the use in accordance with the present invention, the composition could be provided in a form of a mask, a lotion, a cream, an emulsion, a foundation cream, a foundation liquid, a sunscreen, or a bath preparation, but is not limited thereby. That is, the composition could be applied to a subject in need in form as at least one of a mask, a lotion, a cream, an emulsion, a foundation cream, a foundation liquid, a sunscreen, and a bath preparation, but is not limited thereby.

In one embodiment of the present invention, the earthworm protein comprised in the composition is obtained by extracting earthworms with water. In one embodiment, the earthworm protein comprised in the composition is obtained by extracting earthworms with warm water of about 40 to 50° C.

In the use in accordance with the present invention, the earthworm is at least one of Pheretima aspergillum (E. Perrier), Pheretima vulgaris Clen, Eisenia rosea savigny, Eisenia Foetida, Pheretima pectinifera, and Phererima guillelmi.

### BRIEF DESCRIPTION OF THE DRAWINGS

FIG. 1 shows the changes of the concentration of erythema in the skin of a subject, prior to and after applying a conditioning skin composition for 30 minutes, wherein the conditioning skin composition was prepared in accordance with the present invention.

FIG. 2 shows the changes of the size of pores on the skin of a subject, prior to and after applying a conditioning skin composition for 30 minutes, wherein the conditioning skin composition was prepared in accordance with the present invention.

### DESCRIPTION OF THE PREFERRED EMBODIMENTS

The following will describe some of the embodiments of the present invention in detail. However, without departing from the spirit of the present invention, the present invention may be embodied in various embodiments and should not be limited to the embodiments described in the specification.

In addition, unless otherwise indicated herein, the expressions “a,” “an,” “the,” or the like recited in the specification of the present invention (especially in the claims) are intended to include both the singular and plural forms. The term “subject” recited in the specification refers to a mammalian, including human and non-human animals. The term “an effective amount” recited in this specification refers to the amount of the earthworm protein (as dry powders) that can at least partially reduce the concentration of erythema in the skin and/or decrease the size of pores on the skin of a subject.

Earthworm protein recited in this specification refers to an extract of earthworms, which can be obtained by the following processes. The extraction processes of earthworm protein are well known by persons having ordinary skill in the art, so the following extraction processes are provided only for exemplification and the scope of the present invention is not limited thereby.

### Example 1: Preparation of Earthworm Protein

Earthworms were put in water and stirred to wash out the silt, impurities and mucous membrane from the earthworms, until the water runs clear. The earthworms could also be subject to an emesis induction before being washed, so as to discharge the undigested food or impurities from their bodies. Then, the cleaned earthworms were subjected to a sterilization by a trioxygen solution. After the sterilization, the earthworms were taken out, washed again, and then soaked in warm water of 40 to 50° C. for 12 to 16 hours at a weight ratio of earthworms:water=about 1:20 to 1:5, and preferably at a weight ratio of earthworms:water=about 1:12 to 1:8. Thereafter, the earthworms were ground by an attrition grinder to provide a serous fluid. The serous fluid was filtrated by a 50 to 100 mesh filter, and then, the filtrate was collected and centrifuged at 6000 to 14000 rpm for 8 to 28 minutes (both the centrifugation speed and time could be adjusted based on the filtrate volume). After that, the supernatant was collected, and then, filtrated and centrifuged again by the aforementioned conditions. Finally, the supernatant thus obtained was earthworm extract, which was also called as earthworm protein. The earthworm extract could be further concentrated by an ultrafiltration membrane, or be further freeze-dried to powders (i.e., earthworm protein powders).

Depending on the method well known in the field of skin care products, the above earthworm protein could be further prepared as a solution, an emulsion, or a paste that is suitable to be processed into a liniment or an ointment. Alternatively, the earthworm protein could be adsorbed on a usual mask or cotton pad so as to be applied or smeared onto the face. Furthermore, the earthworm protein could be placed in a high-pressure jet bottle so as to be applied as a spray to facilitate the use of the conditioning skin composition over a larger area of skin.

The aforementioned earthworms could be the animals belonging to the phylum Annelida and class Oligochaeta, such as Pheretima aspergillum (E. Perrier), Pheretima vulgaris Clen, Eisenia rosea savigny, Eisenia Foetida, Pheretima pectinifera, or Phererima guillelmi, but is not limited thereby.

### Example 2: Effect of Earthworm Protein on the Concentration of Erythema in Skin

The earthworm protein powders prepared by Example 1 was dissolved in pure water to provide a 0.005 to 0.05 wt % solution, preferably, a 0.015 wt % solution was provided. A dry mask that is free of any ingredient was cut into two pieces, and then, one of the two pieces was soaked in about 10 ml pure water (i.e., control group), the other was soaked in 10 ml pure water containing 0.015 wt % earthworm protein (i.e., earthworm protein group). Thereafter, the masks of the two groups were applied over each half side of face of the subjects for 20 minutes.

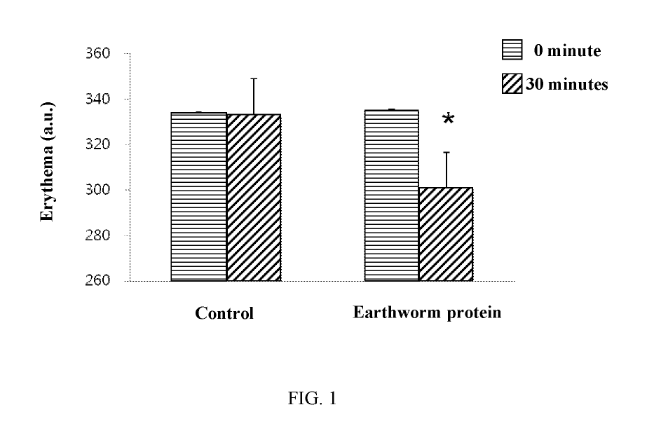

Then, the masks were removed, and the face of each subject was massaged evenly to enhance the absorption of the ingredients in the masks. Thereafter, the skin condition of the subjects was analyzed at the 30^{th} minute counting from the time of applying the masks (the skin condition was also analyzed prior to applying the masks). The skin condition was measured by a MPA multiprobe skin analyzer (purchased from C+K electronic, Germany). The concentration of erythema in skin was measured and calculated by the analyzer via different wavelengths of light being emitted by the probe as well as the principles of light absorption and light reflection. The changes of erythema on skin, prior to and after applying the masks, are shown in FIG. 1.

As shown in FIG. 1, the human clinical trial proves that, the concentration of erythema in the skin of the subjects applying the mask of earthworm protein for 30 minutes was significantly reduced by 10.4%, while no effect was observed from the subjects applying the mask of control group. These results indicate that earthworm protein is effective in calming and reducing skin sensitivity, and thus, can reduce the concentration of erythema in skin significantly.

### Example 3: Effect of Earthworm Protein on the Size of Pores on Skin

The earthworm protein powders prepared by Example 1 was dissolved in a normal saline solution to provide a 0.005 to 0.05 wt % solution, preferably a 0.015 wt % solution was provided. A dry mask free of any ingredient was cut into two pieces, and then, one of the two pieces was soaked in about 10 ml normal saline solution (i.e., control group), the other was soaked in 10 ml normal saline solution containing 0.015 wt % earthworm protein (i.e., earthworm protein group). Thereafter, the masks of the two groups were applied over each half side of face of the subjects for 20 minutes.

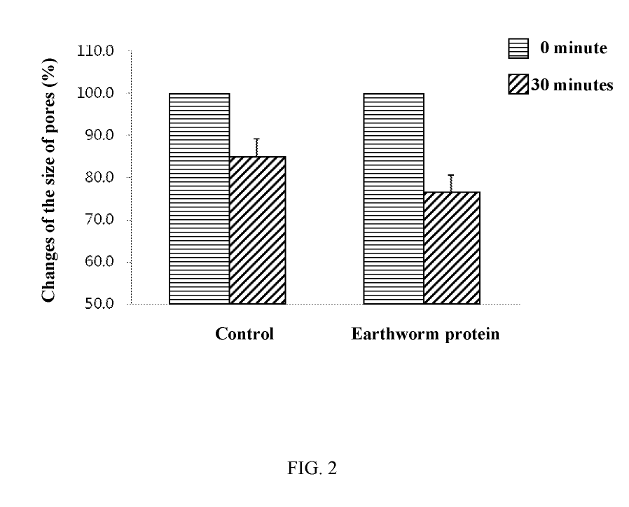

Then, the masks were removed, and the face of each subject was massaged evenly to enhance the absorption of the ingredients in the masks. Thereafter, the skin condition of the subjects was analyzed at the 30^{th} minute counting from the time of applying the masks (the skin condition was also analyzed prior to applying the masks). The skin condition was measured by a skin analyzer, TD (purchased from C+K electronic, Germany). The face skin of each subject was taken a clear photo in a closed film studio by a high-resolution single-lens reflex camera with two light sources (i.e. one is of full spectrum wavelength and the other is of polarized wavelength), and then, the photos were analyzed by a professional software to determine the size of pores on skin. The changes of the size of pores on the skin of the subjects, prior to and after applying the masks, are shown in FIG. 2.

As shown in FIG. 2, the size of pores on the face of the subjects applying the mask of earthworm protein for 30 minutes was significantly decreased by 23.6%. These results prove that the earthworm protein also has an excellent effect on decreasing the size of pores on skin.

Given the above, the earthworm protein is effective in calming, reducing skin sensitivity and alleviating inflammation, and thus, has an outstanding contribution on skin care. Therefore, the earthworm protein can be used in the manufacture of a composition for conditioning skin such as a cosmetic product or a skin care product.

### BRIEF DESCRIPTION OF REFERENCE NUMERALS

Not applicable.

### DEPOSIT OF BIOLOGICAL MATERIAL

Not applicable.

What is claimed is:

1. A method for conditioning skin, comprising applying to a subject in need an effective amount of a composition, wherein the composition comprises earthworm protein.

2. The method as claimed in claim 1, wherein the composition is applied as a solution, an emulsion, or a paste.

3. The method as claimed in claim 1, wherein the concentration of the earthworm protein (as dry powders) in the composition is about 0.005 to 0.05 wt %.

4. The method as claimed in claim 1, wherein the composition is applied to the subject in form as at least one of a mask, a lotion, a cream, an emulsion, a foundation cream, a foundation liquid, a sunscreen, and a bath preparation.

5. The method as claimed in claim 1, wherein the earthworm protein is obtained by extracting earthworms with water.

6. The method as claimed in claim 5, wherein the earthworm protein is obtained by extracting earthworms with warm water of about 40 to 50° C.

7. The method as claimed in claim 5, wherein the earthworm is at least one of Pheretima aspergillum (E. Perrier), Pheretima vulgaris Clen, Eisenia rosea savigny, Eisenia Foetida, Pheretima pectinifera, and Phererima guillelmi.

8. The method as claimed in claim 6, wherein the earthworm is at least one of Pheretima aspergillum (E. Perrier), Pheretima vulgaris Clen, Eisenia rosea savigny, Eisenia Foetida, Pheretima pectinifera, and Phererima guillelmi.

9. The method as claimed in claim 1, wherein the method is for reducing the concentration of erythema on skin and/or decreasing the size of pores on skin.

10. The method as claimed in claim 9, wherein the composition is applied as a solution, an emulsion, or a paste.

11. The method as claimed in claim 9, wherein the concentration of the earthworm protein (as dry powders) in the composition is about 0.005 to 0.05 wt %.

12. The method as claimed in claim 9, wherein the composition is applied to the subject in form as at least one of a mask, a lotion, a cream, an emulsion, a foundation cream, a foundation liquid, a sunscreen, and a bath preparation.

13. The method as claimed in claim 9, wherein the earthworm protein is obtained by extracting earthworms with water.

14. The method as claimed in claim 13, wherein the earthworm protein is obtained by extracting earthworms with warm water of about 40 to 50° C.

15. The method as claimed in claim 13, wherein the earthworm is at least one of Pheretima aspergillum (E. Perrier), Pheretima vulgaris Clen, Eisenia rosea savigny, Eisenia Foetida, Pheretima pectinifera, and Phererima guillelmi.

16. The method as claimed in claim 14, wherein the earthworm is at least one of Pheretima aspergillum (E. Perrier), Pheretima vulgaris Clen, Eisenia rosea savigny, Eisenia Foetida, Pheretima pectinifera, and Phererima guillelmi.
